(19) United States
(12) Patent Application Publication
(10) Pub. No.: US 2011/0059139 A1
(43) Pub. Date: Mar. 10, 2011

(54) METHOD FOR LOADING A MOLECULE INTO A POROUS SUBSTRATE

(76) Inventors: Anna HILLERSTRÖM, Solna (SE); Stefan Wolf, Amberg (DE); Horst Helmut Lux, Neuried (DE)

(21) Appl. No.: 12/555,394

(22) Filed: Sep. 8, 2009

(51) Int. Cl. A61K 9/00; A61K 31/19; A61P 29/00

(52) U.S. Cl. 424/400; 514/568

(57) ABSTRACT

A method for loading a molecule into a porous substrate. The molecule has the formula

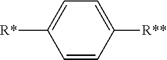

wherein R* is a hydrophobic species and R** is a hydrophilic species which can be Ibuprofen is mixed with mesoporous silica and allowed to contact liquid carbon dioxide for a sufficient period of time to allow the Ibuprofen to load into the pores of the mesoporous silica.

### BACKGROUND OF THE INVENTION

Ibuprofen is an extensively used analgesic and anti-inflammatory drug with fairly low water solubility. Its size of 0.5×1.2×0.8 nm is rather small compared to other drug molecules.

In the treatment of pain symptoms, the controlled delivery of drugs is desirable as it offers advantages over systems where the release of the drug into the patient is relatively instantaneous. Thus an individual taking Ibuprofen may ingest the recommended dosage and later on in a periodic fashion ingest the recommended dosage again.

Various systems have been employed where the controlled release of Ibuprofen is achieved. For example, faujasites and cross-linked polymers have been used as well as various solutions and dispersions. Other porous structure materials besides faujasites can also be used to provide the substance which can hold the Ibuprofen prior to its release. However, the Ibuprofen must be dissolved into a solvent which will deliver the dissolved Ibuprofen into the pores of the porous structural material. Different adsorbents have been employed as the “loading” solvent but they experience the negative result of leaving a solvent residue within the pore structure after loading of the active substance. This disadvantage is unacceptable when the porous structural material is used as a drug delivery device.

Various attempts have been made to load Ibuprofen into a mesoporous substrate. For example, cyclohexane has been used as the loading solvent. Methanol has also been used as the solvent. A low concentration of Ibuprofen in hexane has been employed in achieving a high loading of Ibuprofen in mesoporous silica. Supercritical carbon dioxide has also been employed to load Ibuprofen into mesoporous silica.

The inventors have discovered that liquid carbon dioxide when used as the solvent will not leave the solvent residue in the final material, as well as providing other advantages at loading Ibuprofen into a porous material.

The invention is able to overcome some of the limitations of using supercritical carbon dioxide as liquid carbon dioxide requires less pressure and temperature for loading while achieving similar loading results.

### SUMMARY OF THE INVENTION

The invention provides for a method for loading a molecule having the formula:

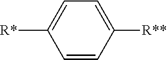

in a porous substrate material comprising contacting the molecule and porous substrate material with liquid carbon dioxide, wherein R* is a hydrophobic species and R** is a hydrophilic species.

R* is a linear or branched alkyl group having from 1 to 6 carbon atoms; R* is a linear or branched alkene group having from 1 to 6 carbon atoms or R* a linear or branched alkyne group having from 1 to 6 carbon atoms.

R** is selected from the group of hydrophilic species selected from the group consisting of halogens;

a group having the formula

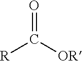

wherein R is selected from the group consisting of the benzene ring with the hydrophobic group R* and a linear or branched group of 0 to 4 carbon atoms connecting the benzene ring to the hydrophilic group,
wherein R′ is selected from the group consisting of hydrogen (carboxyl group) or a linear or branched group of 1 to 4 carbon atoms;
a group having the formula

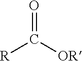

wherein R′ is selected from the group consisting of the benzene ring with the hydrophobic group R* and a linear or branched group of 0 to 4 carbon atoms connecting the benzene ring to the hydrophilic group,
wherein R is selected from the group consisting of hydrogen or a linear or branched group of 1 to 4 carbon atoms;
a group having the formula

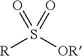

wherein R is selected from the group consisting of the benzene ring with the hydrophobic group R* and a linear or branched group of 0 to 4 carbon atoms connecting the benzene ring to the hydrophilic group,
wherein R′ is selected from the group consisting of hydrogen or a linear or branched group of 1 to 4 carbon atoms;

a group having the formula

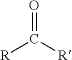

wherein R is selected from the group consisting of the benzene ring with the hydrophobic group R* and a linear or branched group of 0 to 4 carbon atoms connecting the benzene ring to the hydrophilic group,
wherein R′ is selected from the group consisting of hydrogen or a linear or branched group of 1 to 4 carbon atoms;
a group having the formula

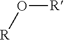

wherein R is selected from the group consisting of the benzene ring with the hydrophobic group R* and a linear or branched group of 0 to 4 carbon atoms connecting the benzene ring to the hydrophilic group,
wherein R′ is selected from the group consisting of hydrogen or a linear or branched group of 1 to 4 carbon atoms;

The invention further provides for a method of loading a molecule having the formula:

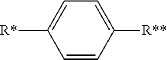

in a porous substrate material comprising the steps of:
a) combining the porous substrate and molecule together;
b) contacting the combination with liquid carbon dioxide; and
c) allowing sufficient time for the molecule to load into the porous substrate material; wherein R* is a hydrophobic species and R** is a hydrophilic species.

R* is a linear or branched alkyl group having from 1 to 6 carbon atoms; R* is a linear or branched alkene group having from 1 to 6 carbon atoms or R* a linear or branched alkyne group having from 1 to 6 carbon atoms.

R** is selected from the group of hydrophilic species selected from the group consisting of halogens;

a group having the formula

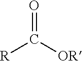

wherein R is selected from the group consisting of the benzene ring with the hydrophobic group R* and a linear or branched group of 0 to 4 carbon atoms connecting the benzene ring to the hydrophilic group,
wherein R′ is selected from the group consisting of hydrogen (carboxyl group) or a linear or branched group of 1 to 4 carbon atoms;
a group having the formula

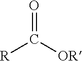

wherein R′ is selected from the group consisting of the benzene ring with the hydrophobic group R* and a linear or branched group of 0 to 4 carbon atoms connecting the benzene ring to the hydrophilic group,
wherein R is selected from the group consisting of hydrogen or a linear or branched group of 1 to 4 carbon atoms;
a group having the formula

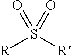

wherein R is selected from the group consisting of the benzene ring with the hydrophobic group R* and a linear or branched group of 0 to 4 carbon atoms connecting the benzene ring to the hydrophilic group,
wherein R′ is selected from the group consisting of hydrogen or a linear or branched group of 1 to 4 carbon atoms;
a group having the formula

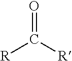

wherein R is selected from the group consisting of the benzene ring with the hydrophobic group R* and a linear or branched group of 0 to 4 carbon atoms connecting the benzene ring to the hydrophilic group,
wherein R′ is selected from the group consisting of hydrogen or a linear or branched group of 1 to 4 carbon atoms;
a group having the formula

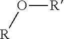

wherein R is selected from the group consisting of the benzene ring with the hydrophobic group R* and a linear or branched group of 0 to 4 carbon atoms connecting the benzene ring to the hydrophilic group,
wherein R′ is selected from the group consisting of hydrogen or a linear or branched group of 1 to 4 carbon atoms.

In either embodiment, ibuprofen is successfully loaded into the porous substrate material using liquid carbon dioxide.

The release of Ibuprofen from mesoporous silica into water is also improved. Fifty percent of the Ibuprofen is released into water within one minute of the mesoporous silica being added to water. This is desirable as a quicker release for an analgesic drug will result in faster relief for the individual taking the analgesic drug.

Ordered mesostructured silica has been employed in applications such as catalysis, adsorption and separation. This material has a tunable pore size in the mesopore range (2 to 50 nm) and a high specific surface area and large pore volume. Typical of this material is the ordered hexagonal MCM-41 with a pore diameter of 3 to 5 nm and a specific area greater than 1000 m²/g and a pore volume greater than 0.7 cm³/g. Due to this unique structure, mesoporous silica material has the potential to act as a drug delivery device.

The liquid carbon dioxide employed in the invention is typically at a temperature of about 20° C. and a pressure of 55±2 bar. However, the liquid CO₂ can be used in ranges of −56° C. to 31° C. and 5.2 bar to 100 bar.

### BRIEF DESCRIPTION OF THE DRAWINGS

FIG. 1 is a graph of the loading capacity of Ibuprofen in mesoporous SiO₂ for three different amounts of Ibuprofen.

FIG. 2 is a graph of the loading capacity of Ibuprofen in mesoporous SiO₂ in CO₂ and in CO₂ and a cosolvent.

### DETAILED DESCRIPTION OF THE INVENTION

The invention is a method for loading a molecule into a porous substrate material using liquid carbon dioxide as a solvent. The molecule can be Ibuprofen and the porous substrate material mesoporous silica.

The loading of Ibuprofen into mesoporous silica, using liquid carbon dioxide as the solvent, with the important end-goal of maximization of the amount of Ibuprofen contained in the pores of the mesostructured silica was studied. The solubility of Ibuprofen in the solvent was considered to be an important factor and consequently the loading efficiencies of pure CO₂ (I) as well as CO₂ (I) mixed with various co solvents have been evaluated. The Ibuprofen-loaded samples were also analyzed with some analytical techniques. The amount of ibuprofen in the mesoporous material was analyzed with a thermogravimetric analyzer (TGA).

TABLE 1
The properties of the mesoporous silica synthesized with a
spray-drying method

| BET | Pore | Mean pore |
|---|---|---|
| surface area | volume | diameter |
| (m²/g) | (cm³/g) | (nm) |
| 1106 | 0.73 | 2.5 |

The mesostructured silica (MCM-41, with a cylindrical mesoporous network) used in the testing was synthesized at YKI, Institute for Surface Chemistry. In brief, the mesoporous particles were synthesized by preparing a precursor solution by mixing tetraethoxysilane (TEOS, Purum, 98%, Fluka) in dilute hydrochloric acid (pH 2) and ethanol (99.7%, Solveco Chemicals AB, Sweden) at room temperature. The cationic surfactant, hexadecyl trimethyl ammonium bromide (CTAB, 95%, Aldrich, Germany), was dissolved in ethanol and then mixed with the hydrolyzed TEOS solution. The mesoporous particles were then formed at room temperature by spraying the solution in a spray drying equipment. This was followed by a calcination step at 550° C. for 4 hours to remove the surfactant templates. The properties of the mesoporous silica are listed in Table 1. The drug molecule in all loading studies in this article was Ibuprofen (>98%, Sigma-Aldrich, Germany). The carbon dioxide used was of industrial grade (99.7%, AGA Gas AB, Sweden) and cosolvents mixed with CO₂ (I) were; cyclohexane (p.a., Merck, Germany), acetone (p.a., Merck, Germany) and methanol (p.a., Merck, Germany).

Loading of Ibuprofen into SiO₂

The mesoporous particles were placed in a bag (fabric of polypropene, PP 3333, permeability 105 DIN, Derma AB, Sweden), which was permeable for the solvent and dissolved Ibuprofen molecules but kept the mesoporous particles in one place in the reactor (see below). The amount of mesoporous particles was 0.1 g in all experiments, while the amount of Ibuprofen was varied for different loading experiments. The particles in the bag and Ibuprofen crystals (thoroughly ground in a mortar before use) were placed in a glass beaker (400 ml), which was placed in an in-house built stainless steel reactor (1.7 L) with two sapphire glass windows. The reactor was first pressurized with carbon dioxide and thereafter 200 ml CO₂ (I) or CO₂ (I)+5 mol-% cosolvent was introduced into the glass beaker at 20° C. and 55±2 bar. During the loading of Ibuprofen into the mesoporous material, the solution was gently stirred at constant speed with a magnetic bar. After the loading period (15 minutes to 18 hours), the reactor was depressurized and the bag containing the mesoporous particles with Ibuprofen was retrieved.

Thermogravimetric Analysis (TGA)

The samples were characterized with TGA using a TGA 7 instrument (Perkin Elmer Inc., USA). The temperature program used consisted of an initial part with a heating rate of 20° C./min from 20° C. to 95° C., followed by an isothermal pause for 60 minutes at 95° C., and finally heating from 95° C. up to 800° C. at a heating rate of 2° C./min. All TGA measurements were performed under 20 ml/min flow of N2 gas.

The solubility of Ibuprofen in CO₂ (I) and CO₂ (I) with 5 mol % of cosolvent was determined by visual inspection of sample mixtures of the solvent(s) and Ibuprofen at room temperature and 55±2 bar, and the solubility limit was considered to be reached when particles of Ibuprofen were visible. The dissolution of Ibuprofen appeared to occur within less than 30 minutes but the solubility test was performed for 9 hours to ascertain an equilibrium value. The solubility tests are summarized in Table 2.

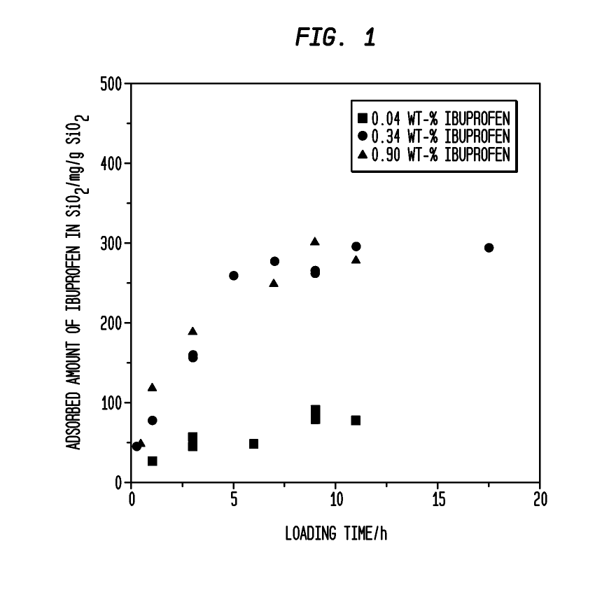

The results from experiments with loading of Ibuprofen in mesoporous particles using CO₂ (I) are summarized in FIG. 1.

TABLE 2
The solubility limit of Ibuprofen in
CO₂, (I) and CO₂ (I) + cosolvent

| | Solubility | Solubility |
|---|---|---|
| | limit | parameter of |
| Solvent | (wt-%) | cosolvent (MPa^{1/2}) |
| CO₂ (I) | 0.20-0.25 | |
| CO₂ (I) + cyclohexane | 1.0-1.5 | 16.8 |
| CO₂ (I) + acetone | 1.5-2.0 | 20.3 |
| CO₂ (I) + methanol | >2.0 | 29.7 |

The solubility parameter for CO₂ (I) is 12 MPa^{1/2}

where the adsorbed amount of Ibprofen in the particles were measure by TGA. The loading time and concentration of Ibuprofen in the CO₂ (I) was varied in the experiments and each experiment is represented by one point in the graph in FIG. 1. Three different amounts of Ibuprofen in 200 ml CO₂ (I) were evaluated: one giving a concentration below the solubility limit (0.04 wt-%) and two amounts with an excess of Ibuprofen present (undissolved) in the CO₂ (I) (nominal concentrations: 0.34 wt-% and 0.90 wt-%).

Firstly, as can be seen from FIG. 1, several hours (7 to 12 hours) were needed to reach the maximum (equilibrium-) loading level, both in the experiments at low and at higher concentrations of Ibuprofen. At least two factors may be relevant for influencing the time to reach the equilibrium: i) time for dissolution and ii) diffusion of dissolved Ibuprofen into the particles. As the dissolution of Ibuprofen in the solvent appeared to be very fast, as observed when performing the above solubility tests, it is theorized that Ibuprofen diffusion, and not Ibuprofen dissolution, is the rate-limiting step for loading of the mesoporous particles in this system.

Secondly, a difference in maximum loaded amount was observed depending on whether the solution was saturated with Ibuprofen, which resulted in a high loading, close to 300 mg Ibuprofen/g SiO₂, or if the concentration of Ibuprofen was below this level, which then gave a much lower loading as seen in FIG. 1.

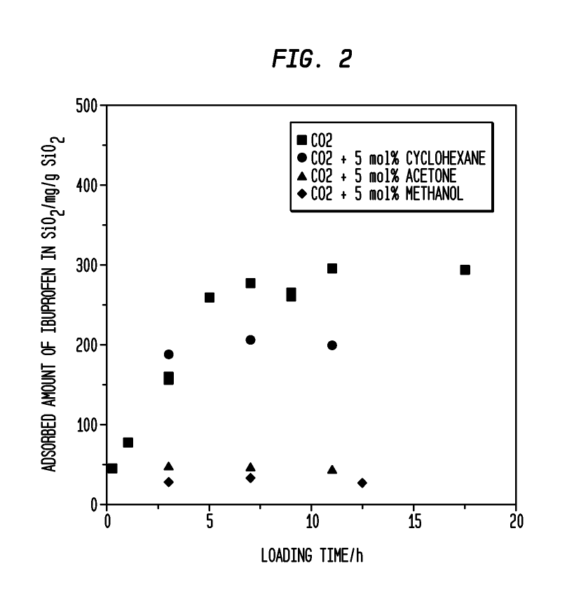

Similar experiments were performed by using 0.34 wt-% Ibuprofen in CO₂ (I) and a cosolvent (5 mol-%), as shown in FIG. 2. However, the addition of a cosolvent did not improve the maximum adsorbed amount of Ibuprofen in the mesoporous particles. On the contrary, using cyclohexane resulted in a slight decrease in the adsorbed amount compared to pure CO₂ (I). The maximum value in the case of CO₂ (I)+cyclohexane was approximately 200 mg Ibuprofen/g SiO₂, compared to 300 mg Ibuprofen/g SiO₂ that was obtained using only CO₂ (I). For acetone and methanol as cosolvents, a large decrease of the adsorbed amount of Ibuprofen in the mesoporous particles was observed. Only approximately 50 mg Ibuprofen/g SiO₂ was loaded into the particles in both cases.

The solubility of Ibuprofen in CO₂ (I) is low (0.20-0.25 wt %), but despite this, a high loading capacity of Ibuprofen into the mesostructured silica (300 mg Ibuprofen/g SiO₂) can be achieved by exposing mesoporous silica particles to a saturated solution of Ibuprofen for several hours (7 to 12 hours).

When introducing a more polar cosolvent to liquid carbon dioxide, more Ibuprofen is dissolved. This results in a lower loading capacity of Ibuprofen into the pores than when using CO₂ (I) alone. In cases where the cosolvent can form hydrogen bonds with mesoporous material there will be a competition between the cosolvent and Ibuprofen to adsorb on the SiO₂ surface. This will result in a lowering of the loading capacity of Ibuprofen, at the conditions studied in this article, where the cosolvents were present in much higher concentration than Ibuprofen, and were also smaller in size (quicker diffusion).

Moreover it has been shown by using X-ray Powder Diffraction (XRPD) that the loaded Ibuprofen molecules are in an amorphous state, most likely due to the fact that the pores in the mesoporous silica are too narrow for crystallization of Ibuprofen to occur.

Finally, it has been demonstrated that the potential of using CO₂ (I) for loading of Ibuprofen into mesoporous SiO₂ in the near critical region, avoiding the use of supercritical CO₂, which would require a more energy-intensive process.

While this invention has been described with respect to particular embodiments thereof, it is apparent that numerous other forms and modifications of the invention will be obvious to those skilled in the art. The appended claims in this invention generally should be construed to cover all such obvious forms and modifications which are within the true spirit and scope of the invention.

Having thus described the invention, what we claim is:

1. A method for loading a molecule having the formula: 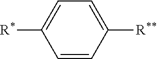
in a porous substrate material comprising contacting said molecule and porous substrate material with liquid carbon dioxide, wherein R* is a hydrophobic species and R** is a hydrophilic species.

2. The method as claimed in claim 1 wherein R* is a linear or branched alkyl group having from 1 to 6 carbon atoms.

3. The method as claimed in claim 1 wherein R* is a linear or branched alkene group having from 1 to 6 carbon atoms.

4. The method as claimed in claim 1 wherein R* is a linear or branched alkyne group having from 1 to 6 carbon atoms.

5. The method as claimed in claim 1 wherein R** is a halogen.

6. The method as claimed in claim 1 wherein R** is a group having the formula 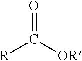
wherein R is selected from the group consisting of the benzene ring with the hydrophobic group R* and a linear or branched group of 0 to 4 carbon atoms connecting the benzene ring to the hydrophilic group,
wherein R′ is selected from the group consisting of hydrogen (carboxyl group) or a linear or branched group of 1 to 4 carbon atoms.

7. The method as claimed in claim 1 wherein R** is a group having the formula 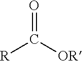
wherein R′ is selected from the group consisting of the benzene ring with the hydrophobic group R* and a linear or branched group of 0 to 4 carbon atoms connecting the benzene ring to the hydrophilic group,
wherein R is selected from the group consisting of hydrogen or a linear or branched group of 1 to 4 carbon atoms.

8. The method as claimed in claim 1 wherein R** is a group having the formula 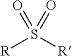
wherein R is selected from the group consisting of the benzene ring with the hydrophobic group R* and a linear or branched group of 0 to 4 carbon atoms connecting the benzene ring to the hydrophilic group,
wherein R′ is selected from the group consisting of hydrogen or a linear or branched group of 1 to 4 carbon atoms.

9. The method as claimed in claim 1 wherein R** is a group having the formula 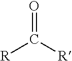
wherein R is selected from the group consisting of the benzene ring with the hydrophobic group R* and a linear or branched group of 0 to 4 carbon atoms connecting the benzene ring to the hydrophilic group,
wherein R′ is selected from the group consisting of hydrogen or a linear or branched group of 1 to 4 carbon atoms.

10. The method as claimed in claim 1 wherein R** is a group having the formula 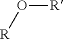
wherein R is selected from the group consisting of the benzene ring with the hydrophobic group R* and a linear or branched group of 0 to 4 carbon atoms connecting the benzene ring to the hydrophilic group,
wherein R′ is selected from the group consisting of hydrogen or a linear or branched group of 1 to 4 carbon atoms.

11. The method as claimed in claim 1 wherein said molecule is Ibuprofen.

12. The method as claimed in claim 1 wherein said porous substrate material is mesoporous silica.

13. The method as claimed in claim 1 wherein said liquid carbon dioxide is at a temperature of −56° C. to 31° C. and a pressure of 5.2 bar to 100 bar.

14. The method as claimed in claim 1 wherein said liquid carbon dioxide contacts said drug molecule for a period of about 1 min to 48 hours.

15. The method as claimed in claim 1 wherein said molecule is for human consumption.

16. The method as claimed in claim 11 wherein said Ibuprofen has a concentration of 0.01 to 0.9 wt.-% in the liquid carbon dioxide.

17. The method as claimed in claim 12 wherein said Ibuprofen is present in said mesoporous silica in an amount of 20 mg to about 300 mg Ibuprofen per gram of mesoporous silica.

18. A method of loading a molecule having the formula: 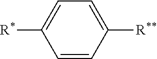
wherein R* is a hydrophobic species and R** is a hydrophilic species
in a porous substrate material comprising the steps of:
a) combining said porous substrate and said molecule together;
b) contacting the combination with liquid carbon dioxide; and
c) allowing sufficient time for said molecule to load into said porous substrate material.

19. The method as claimed in claim 18 wherein R* is a linear or branched alkyl group having from 1 to 6 carbon atoms.

20. The method as claimed in claim 18 wherein R* is a linear or branched alkene group having from 1 to 6 carbon atoms.

21. The method as claimed in claim 18 wherein R* is a linear or branched alkyne group having from 1 to 6 carbon atoms.

22. The method as claimed in claim 18 wherein R** is a halogen.

23. The method as claimed in claim 18 wherein R** is a group having the formula 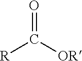
wherein R is selected from the group consisting of the benzene ring with the hydrophobic group R* and a linear or branched group of 0 to 4 carbon atoms connecting the benzene ring to the hydrophilic group,
wherein R′ is selected from the group consisting of hydrogen (carboxyl group) or a linear or branched group of 1 to 4 carbon atoms.

24. The method as claimed in claim 18 wherein R** is a group having the formula 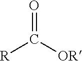
wherein R′ is selected from the group consisting of the benzene ring with the hydrophobic group R* and a linear or branched group of 0 to 4 carbon atoms connecting the benzene ring to the hydrophilic group,
wherein R is selected from the group consisting of hydrogen or a linear or branched group of 1 to 4 carbon atoms.

25. The method as claimed in claim 18 wherein R** is a group having the formula 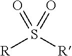
wherein R is selected from the group consisting of the benzene ring with the hydrophobic group R* and a linear or branched group of 0 to 4 carbon atoms connecting the benzene ring to the hydrophilic group,
wherein R′ is selected from the group consisting of hydrogen or a linear or branched group of 1 to 4 carbon atoms.

26. The method as claimed in claim 18 wherein R** is a group having the formula 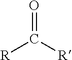
wherein R is selected from the group consisting of the benzene ring with the hydrophobic group R* and a linear or branched group of 0 to 4 carbon atoms connecting the benzene ring to the hydrophilic group,
wherein R′ is selected from the group consisting of hydrogen or a linear or branched group of 1 to 4 carbon atoms.

27. The method as claimed in claim 18 wherein R** is a group having the formula 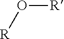
wherein R is selected from the group consisting of the benzene ring with the hydrophobic group R* and a linear or branched group of 0 to 4 carbon atoms connecting the benzene ring to the hydrophilic group,
wherein R′ is selected from the group consisting of hydrogen or a linear or branched group of 1 to 4 carbon atoms.

28. The method as claimed in claim 18 wherein said molecule is Ibuprofen.

29. The method as claimed in claim 18 wherein said porous substrate material is mesoporous silica.

30. The method as claimed in claim 18 wherein said liquid carbon dioxide is at a temperature of −56° C. to 31° C. and a pressure of 5.2 bar to 100 bar.

31. The method as claimed in claim 18 wherein said liquid carbon dioxide contacts said molecule for a period of about 1 min to 48 hours.

32. The method as claimed in claim 18 wherein said molecule is for human consumption.

33. The method as claimed in claim 28 wherein said Ibuprofen has a concentration of 0.01 to 0.9 wt.-% in the liquid carbon dioxide.

34. The method as claimed in claim 29 wherein said Ibuprofen is present in said mesoporous silica in an amount of 20 mg to about 300 mg Ibuprofen per gram of mesoporous silica.
